(19) United States
(12) Patent Application Publication
(10) Pub. No.: US 2020/0165559 A1
(43) Pub. Date: May 28, 2020

(54) Ejector Equipped Fermenter

(71) Applicant: Novozymes A/S, Bagsvaerd (DK)

(72) Inventors: Anders Peter Jensen, Bagsvaerd (DK); John Bach, Bagsvaerd (DK)

(73) Assignee: Novozymes A/S, Bagsvaerd (DK)

(21) Appl. No.: 16/636,768

(22) PCT Filed: Aug. 7, 2018
(86) PCT No.: PCT/EP2018/071310

§ 371 Date: Feb. 5, 2020

(30) Foreign Application Priority Data
Aug. 7, 2017 (EP) 17185045.6

(51) Int. Cl. C12M 1/00; C12M 1/12; C12M 1/34; C12N 9/24; C12N 9/34; C12P 21/02; C12N 9/54

(52) U.S. Cl. CPC C12M 29/06; C12M 37/00; C12M 41/00; C12M 29/18; C12Y 302/01003; C12N 9/248; C12N 9/2428; C12P 21/02; C12N 9/54; C12M 21/14

(57) ABSTRACT

Disclosed is a fermenter comprising one or more two phase injectors for providing oxygen for the fermentation and a circulation loop, circulating the fermentation broth and providing liquid for the two-phase injectors. Further disclosed is a fermentation plant comprising one or more fermenters of the invention and utility for the fermenters. Also disclosed is the use of the fermenters for the production of a fermentation product such as an enzyme.

### FIELD OF THE INVENTION

The present invention relates to fermenter for growing microorganisms for the production of a product. In particular, the invention relates to fermenters for fermenting microorganisms for the production of a fermentation product such as a protein.

### BACKGROUND OF THE INVENTION

Fermentation has been practiced in many different industries for many different purposes. Typically, a fermentation broth comprising the necessary nutrients is provided in a fermenter and a given microorganism is added to the fermentation broth and the fermentation is carried out under predetermined conditions whereby the given microorganism produces a desired product.

For high oxygen demanding fermentation an often used fermenter is a stirred tank fermenter (STF), basically consisting of a closed tank equipped with a stirrer consisting of a stirrer shaft and one or more impellers. Oxygen for the fermentation is typically delivered at a point below the impeller in order to secure that air bubbles are closely mixed with the fermentation broth securing a good oxygen transfer. Agitation also provide smaller bubbles which further contributes to a good oxygen transfer.

Another design for fermenters for aerobic fermentations is bubble columns where air is delivered in the lower part of the fermenter typically using a sparger and oxygen is transferred to the fermentation broth while bubbles rise to the top of the tank.

Zlokarnik, (1985) (Tower-Shaped Reactors for aerobic biological waste water treatment, page 537-569 in Biotechnology, Volume 2. Fundamentals of Biochemical Engineering. Rehm and Reed (editors), VCH Verlagsgesellschaft mbH, D-6940 Weinhein, Germany, 1985) discloses waste water treatment plants equipped with injectors, two-phase nozzles, for delivering oxygen for the waste water purification operation.

RU 2580646 discloses a fermentation plant for urethan assimilating microorganisms comprising a column fermenter and two reactors as well as inlet and outlet tubes connecting the fermenter and the reactors in a functional way.

EP 0 916724 discloses a fermentation tank comprising two oppositely directed inlet, located in the mitter part of the fermenter. It is described that it is important that the streams from two inlets and injected with different velocities and that the inlets are opposing each other the generate high shearforces in the impact zone.

CN 102731417 discloses an emulsion-type aerobic fermenter including a gas-liquid mixing tank and circulating means located inside the tank.

### SUMMARY OF THE INVENTION

The invention provides a fermenter for high-oxygen demanding fermentation comprising a tank, one or more two-phase injectors for supplying oxygen, at least one loop circulating fluid from the fermenter and providing liquid for the two-phase injectors, one or more inlets and one or more outlets.

Preferably the fermenter is sterilisable and CIP cleanable.

The invention further provides a fermentation plant comprising one of more fermenters of the invention, means for driving the flow in the circulation loops, supply of substrate, nutrients and air, means for analysing the readings of the probes connected to the fermenter, means for regulating pH and temperature and means for delivering additional compounds such as defoaming agents and other compounds required during the fermentation.

Further the invention provides the use of the fermenters or the fermentation plant of the invention for growing one or more cells for the production of one or more fermentation products, such as proteins, in particular enzymes.

### BRIEF DESCRIPTION OF DRAWINGS

FIG. 1 shows a fermenter according to the invention.

FIG. 2 shows the distribution of air bubbles using a two-phase injector.

FIG. 3 shows the arrangement of nozzles in the fermenter described in example 1.

FIG. 4 shows a graph disclosing the relative titer development of cellulose degrading enzyme as a function of fermentation time. The triangles show the data for the nozzle fermenter compared with data from a corresponding fermentation using a stirred tank reactor shown with circles.

FIG. 5 shows the Master-Slave fermenter setup.

### DETAILED DESCRIPTION OF THE INVENTION

The present invention relates to a fermenter comprising a tank provided with one or more two-phase injectors connected to an air supply and a liquid supply, a circulation loop for circulating the fermentation broth and providing liquid to the two-phase injectors; one or more inlets for nutrients, inoculants, pH regulating agents, foam regulating agents etc., and one or more outlets for samples, fermentation broth, spent air etc., wherein the fermenter is sterilisable or CIP cleanable to reduce the germ number to a predetermined low number.

The volume of the fermenter is not decisive but the invention can be applied to fermenters having a volume of at least 50 liters, preferably at least 100 liters, preferably at least 500 liters, preferably at least 1000 liters, preferably at least 5000 liters, preferably at least 10000 liters, preferably at least 25000 liters, preferably at least 50000 liters, preferably at least 100000 liters or at least 250000 liters.

The fermenter may be provided with means for cooling, such as a cooling jacket, a spiral of cooling tubes or an external cooler. Means for cooling a fermenter is known in the art and such means for cooling as known in the art will also useable according to the present invention.

The fermenters of the invention are also called “nozzle fermenters” and this term is used herein for fermenters of the invention.

The two-phase injectors used according to the present invention are two-phase nozzles where the kinetic energy of the liquid propulsion breaks up the gas into very fine bubbles. Thus, the two phase injectors are driven by a liquid stream that enters the injector where it meet the air/oxygen in a mixing area/mixing chamber where it produces a dispersion of fine gas bubbles in the liquid phase and the dispersion leaved the injector, via an orifice or nozzle, as a stream of finely dispersed gas bubbles in the liquid. Such two-phase injectors are in the art also known as injectors, ejectors, eductors, venturi ejectors, nozzles etc. Several designs of such two phase injectors are known in the art and the present invention is not limited to any particular design. Examples of two-phase injectors are disclosed in U.S. Pat. Nos. 4,098,851 and 4,162,970.

Two-phase injectors have typically been made on polypropylene and used in waste treatment plants (Zlokarnik (1985) supra) but for the present invention it is important that the injectors are made in a material that can be repeatedly sterilized or CIP cleaned and are not susceptible to scratches and crevices wherein germs could be located and in this way survive heat treatment or cleaning of the fermenter and consequent compromise the sterility or low germ number required in many industrial fermentation processes. The injectors are preferably made in a non-corrosive metal such as steel more preferably stainless steel.

In use the two-phase injectors are connected to an air/oxygen supply and a liquid supply and for a stream of fine gas bubbles dispersed in the liquid phase that gradually is mixed with the reminder of the fermentation broth. The design of the two-phase injector may determine the shape of the dispersed stream, whether it is wide, small, round, flat, fan shaped or other forms.

The air/oxygen supply may be any gas mixtures comprising oxygen in a sufficient amount to secure a satisfactory oxygen transfer to the fermentation broth, typically atmospheric air optionally enriched for oxygen by mixing atmospheric air with pure oxygen. The skilled person will appreciate that the higher the oxygen content is in the air/oxygen supply the higher oxygen transfer is possible, but since costs for additional oxygen is considerable, atmospheric air is the preferred air/oxygen supply. The air supply should be sterilized before being delivered to the fermenter. Methods for sterilizing air are known in the art and such methods may also be applied to the present invention.

In addition to delivering oxygen to the fermentation broth the streams ejected from the two-phase injectors also generate movement and mixing of the fermentation broth, and since the fermenters of the invention do not have any stirrers the movement of the fermentation broth is only driven by the two-phase injectors.

The one or more two-phase injectors are placed in the lower part of the fermenter, so that the oxygen from the bubbles ejected from the injectors can be transferred to the liquid phase while the bubbles raise to the top of the liquid. If more than one two-phase injectors are provided they should be arranged so their injected streams do not collide with each others. In some embodiments the air/oxygen supply for the two-phase injectors is provided by one or more ring formed tubes or pipes placed in the lower part of the fermenter and the liquid for the two-phase injectors is provided by one or more ring formed tubes or pipes placed in the lower part of the fermenter e.g. arranged so that the one or more two-phase injectors are bridging between the one or more ring formed tube or pipe for the air supply and the one or more ring formed tube or pipe providing the liquid supply.

In one preferred embodiment at least one of the one or more two phased injectors are arranged so it delivers the stream of gas/liquid dispersion in an downwards direction towards the bottom of the fermenter. This will secure that also the lower part of the fermenter becomes aerated and provides mixing and movement also to the section of the fermenter below the two phase injectors, and thereby securing that this part of the fermenter is productive.

The angle of the stream ejected from the at least one of the one or more two phase injectors should be selected to secure a good aeration and mixing of the fermentation broth in the fermenter. Preferably the at least one of the one or more two phase injectors should be arranged to direct the flow up, preferably in an angle to a horizontal plane in the range of 10° to 90°, preferably in the range of 30° to 80°, preferably in the range of 30° to 75°, more preferred in the range of 40° to 60° and most preferred around 45° such as an angle of 45°.

In one embodiment the fermenter comprises at least two two-phase injectors wherein one two-phase injector directs the stream downwards at an angle to a horizontal plane of 15-75 at 45° and placed parallel to the fermenter wall creating a rotating flow pattern in the bottom part of the fermenter, in order to secure a good aeration and productivity of this part as well. In a preferred embodiment the fermenter comprises at least one two-phase injector ejecting a stream going up and at least one two-phase injector ejecting a stream going down.

The fermenter should be provided with a sufficient number of two-phase injectors to provide the necessary oxygen supply for the fermentation. The fermenter should equipped with a sufficient number of two-phase injectors to deliver at least 400 mg O₂ per hour per kg fermentation broth (mgO₂/kg/hr), preferably at least 500 mgO₂/kg/hr, preferably at least 600 mgO₂/kg/hr, preferably at least 700 mgO₂/kg/hr, e.g. at least 800 mgO₂/kg/hr, e.g. at least 900 mgO₂/kg/hr, e.g. at least 1000 mgO₂/kg/hr, e.g. at least 1200 mgO₂/kg/hr, e.g. at least 1500 mgO₂/kg/hr, e.g. at least 2000 mgO₂/kg/hr, e.g. at least 3000 mgO₂/kg/hr, e.g. at least 4000 mgO₂/kg/hr, e.g. at least 5000 mgO₂/kg/hr, e.g. at least 6000 mgO₂/kg/hr, e.g. at least 7000 mgO₂/kg/hr, e.g. at least 8000 mgO₂/kg/hr and most preferred at least 10000 mgO₂/kg/hr.

The number and the size of two-phase injectors should be determined depending on the amount of oxygen that should be transferred to the fermentation broth. Such two-phase injectors are known in the art and the amount of air and liquid required to achieve a desired oxygen supply can be calculated by the skilled person using the teachings of the art, e.g. as disclosed in: K. Israelsen (2016) Development of a glucose oxidase method for mass transfer characterization in a bioreactor, Master Thesis, Department of Chemical and Biochemical Engineering, Technical University of Denmark (incorporated by reference).

In some embodiments the fermenter comprises at least 0.04 two-phase injector per m³ fermenter volume (=1 injector per 25 m³), preferably at least 0.05 two-phase injector per m³ fermenter volume, preferably at least 0.075 two-phase injector per m³ fermenter volume, preferably at least 0.1 two-phase injector per m³ fermenter volume, preferably at least 0.15 two-phase injector per m³ fermenter volume, preferably 0.20 two-phase injector per m³ fermenter volume and most preferred at least 0.25 two-phase injector per m³ fermenter volume. Typically the number of two-phase injectors is in the range on 0.05 to 0.5 injectors per m³ fermenter volume, preferably in the range of 0.1 to 0.25 injectors per m³ fermenter volume.

The circulation loop removes fermentation broth from the fermenter and returns it to the fermenter at least partially via the two-phase injectors. The circulation loop is connected with a pump to drive the flow in the loop and deliver the necessary flow for the two-phase injectors. The circulation loop may be provided with one or more sensors for controlling the conditions in the fermenter, such as temperature probes and pH probes, and it may be provided with a number of inlets e.g. for pH regulating agent, foam controlling agents, nutrients, or for inoculation of the fermenter; and a number of outlets; e.g. for harvest or sampling.

The flow in the circulation loop should be selected so it is sufficient to drive the two-phase injectors provided in the fermenter. In some embodiments, the flow in the circulation loop is in the range of 5-20 m³/h/number of two-phase injectors depending on the design of the two-phase injectors, such as in the range of 8-20 m³/h/number of two-phase injectors, preferably 10-15 m³/h/number of two-phase injectors, such as around 12.5 m³/h/number of two-phase injectors.

The inlets and outlets to the fermenter and circulation loop should be designed in a way that secure that the sterility of the fermenter is maintained. This is all known in the art and such solution as known in the art will also be useable according to the present invention.

In one preferred embodiment the circulation loop is provided with one or more sensors for determining the conditions in the fermentation broth, such as temperature and pH probes; inlets for pH regulating agent and process aids, such as defoaming agents; inlets for nutrients and inlets for inoculation of the fermenter. In this embodiment the utilities can be separated from the fermenter and e.g. be provided from existing installation at the site or established next to the fermenter e.g. in a separate room, building or facility. This reduces the investment for establishment of new fermenters and further offers the possibility that the utilities for several fermenters, such as pumps for the circulation loop, means for generating sterile air/oxygen for the fermentation, substrate, nutrients, pH regulating agents, foam regulating agents etc. can be arranged together and to some extend shared between two or more fermenters.

The fermenter should also be provided with means for inoculating the fermenter. In one embodiment the means for inoculation is a seed fermenter, i.e. a smaller fermenter designed for preparing seed material for the fermenter wherein the production of the desired fermentation process takes place, often called the main fermenter. The seed fermenter has typically a volume in the range of 5-25% of the main fermenter.

Often two or more seed fermenters may be provided in a series wherein each fermenter has a volume of 5-25% of the next fermenter until the last seed fermenter having a volume of 5-25% of the main fermenter. Such an arrangement is also called a seed train.

The seed fermenter may be placed immediately next to the main fermenter and the inoculation may be done directly via an inlet to the main fermenter, or the seed fermenter may be placed separate from the main fermenter and the inoculation take place via an inlet into the circulation loop.

In other embodiments, the fermenter may not be provided with a seed fermenter. In such a configuration may the fermenter be inoculated seed material directly from the microbiology laboratory.

In other embodiments in a fermentation plant comprising two or more fermenters according to the invention, may one fermenter serve as seed fermenter for two or more similar fermenters.

For use the fermenter is connected to the necessary supplies as known in the area, such as substrate, water, pH regulating agents, defoaming agents, air supply, pumps etc. The fermenter and the necessary supply equipment is in this application and claims called the fermentation plant.

The fermentation plant should be sterilisable or at least prepared in a way so it can be CIP cleaned, i.e. cleaned using a method that reduces the germ number to a sufficient low number to prevent uncontrolled and/or unexpected growth of other cells than the desired cells in the fermenters. Typically, fermenters are sterilized using steam at high pressure and temperature e.g. treating the plant with steam at a temperature of 120-140° C., a pressure of 2-5 bars for a period of 20-60 minutes.

The sterilization procedure used according to the invention should be selected using parameters required to reduce the number of germs by a factor of at least 10⁸, preferably at least 10⁹, preferably at least 10¹⁰, preferably at least 10¹², preferably at least 10¹⁴, preferably at least 10¹⁵, most preferred at least 10¹⁷.

One preferred method for sterilizing the fermenter is by heat sterilization, typically treating the fermenter under autoclaving conditions, such as 120° C. for 20 minutes by steam injections. For substrates containing sediments a longer sterilization time may be required e.g. up to 120 minutes.

CIP (cleaning in place) is a method of cleaning the interior surfaces of pipes, vessels, process equipment and associated fittings without disassembly. The term is commonly used for cleaning bioreactors, fermenters, mix vessels, and other equipment used in biotech manufacturing, pharmaceutical manufacturing and food and beverage manufacturing.

CIP cleaning procedures typically uses a combination of heat, chemical action, and turbulent flow.

The CIP cleaning procedures used according to the invention should be selected using parameters required to reduce the number of germs by a factor of at least 10⁶, preferably at least 10⁷ most preferred at least 10⁸.

The nozzle fermenters of the invention may be generated in any dimension according to the intended use. For fermenting microorganisms for the production of a desired product such as an enzyme preferred dimensions are listed in table 1 below:

TABLE 1

| | Preferred | |
|---|---|---|
| Parameter | Range | Comments |
| Fermenter Volume | 5-1000 | |
| (m³) | | |
| Nozzles per volume | 0.1-0.3 | For present nozzle design. |
| (in m³) | | Depends on Nozzle |
| | | dimensions and design |
| Height (m) | 5-15 | A tall fermenter is optimal |
| | | due to better utilization of |
| | | oxygen. |
| Diameter (m) | 1-10 | |
| Air Pressure (barg) | 1.0-2.0 | Depends on Liquid Height |
| Aeration - VVM | 0.1-1.0 | Depends on Process |
| (volume/fermenter | | Requirements |
| volume/min) | | |
| Liquid Circulation | 0.5-5.0 | |
| (volume/fermenter | | |
| volume/h) | | |

The fermenters according to the invention has several benefits compared with traditional stirred fermenters. First, the fermenters according to the invention are significantly cheaper to construct, in part because there is no need for stirrer and the engine driving the stirrer, but also because the fermenter can be made in lighter materials because the fermenter no longer need to bear the stirrer engine or endure the trembles and forces that inevitable results from arranging and operating an engine and stirrer arrangement on top of a fermenter. Further the fermenter of the invention does not require a building because the necessary utilities can be arranged in a container next to the fermentation tank, and further, by moving most equipment from the tank top to a container enables sharing equipment between two or more fermenters which also contribute to reducing the cost for the individual fermenter. Further, the fermenter design according to the invention allows the formation of very large fermenters because of the simple set-up and also because there is no need to scale up a large agitator.

Still another benefit, the fermenters according to the invention is cheaper in operation than a corresponding stirred tank reactor because the energy consumption is significantly lower. This is in a large extent because the energy required for the pump driving the circulation in the circulation loop according to the present invention is significantly lower than the energy required to drive the stirrer in a corresponding stirred tank fermenter.

Finally, because you don't have an engine running the stirrer there is no need for cooling devices for removing the heat generated by the engine, reducing both construction and operational costs.

Thus, the fermenters according to the invention are significantly cheaper to construct and also requires less energy for operation compared with a stirred tank reactor and are therefore from an economical point of view an attractive alternative to the stirred tank reactor despite the fact that the yield often is a little lower using the fermenters according to the invention.

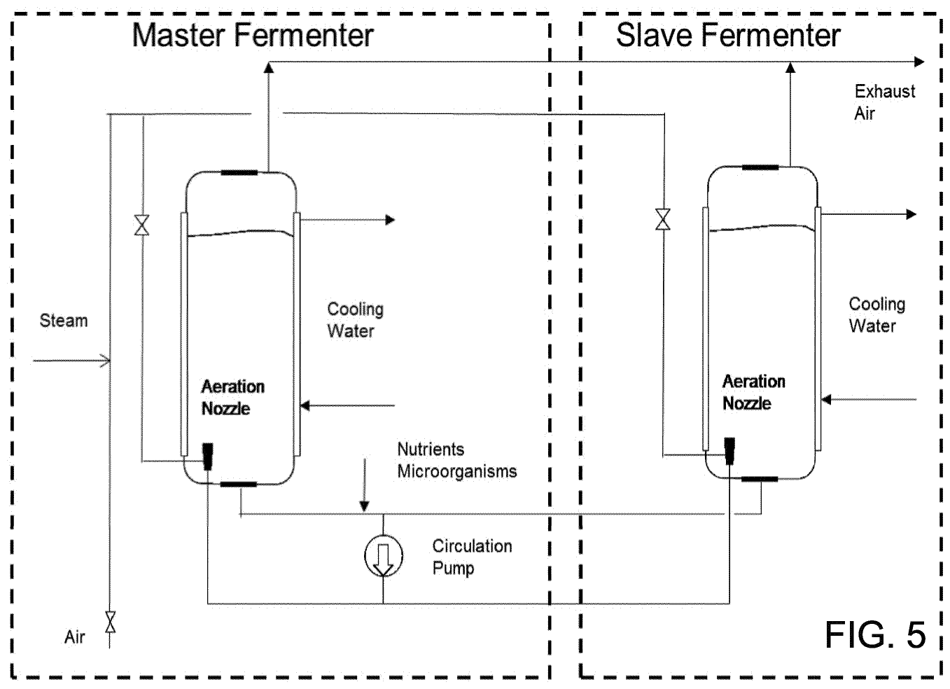

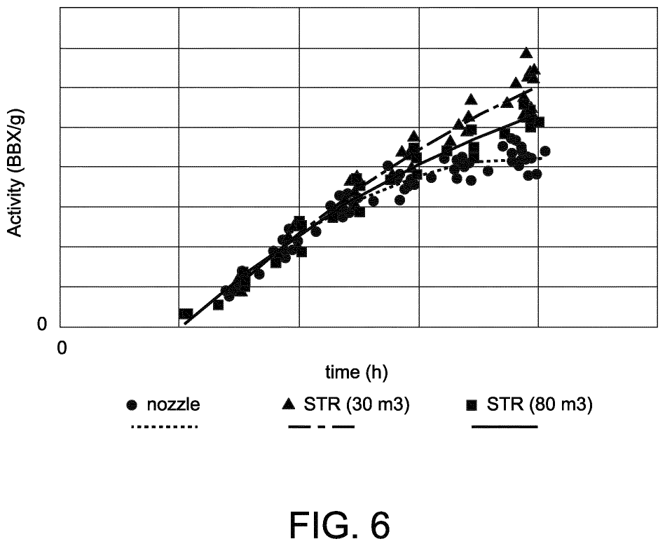

In one preferred embodiment two or more fermenters may be connected via the circulation loops so the fermentation broth is mixed and circulated in two or more fermenters, also called master slave setup. In this embodiment all the instrumentation, control and shared equipment, such as pH control, foam regulation etc; are done on the first fermenter (the master fermenter) and the second and further fermenters (slave fermenters) is/are basically passive units operated in parallel with and controlled via the first fermenter (Master). The slave master concept with one master and one slave fermenter is disclosed in FIG. 5.

It has surprisingly been found that fermentations in a master-slave setup can be run and controlled essentially as if the fermentation was performed in the master fermenter alone.

In one embodiment the master slave set-up contains one master fermenter and one slave fermenter, in other embodiments the master slave set-up comprises one master fermenter and two or more slave fermenters, such as 2, 3, 4, 5, or even more slave fermenters.

The dimensions of the slave fermenter may be identical to or different of the dimensions of the master fermenter, however, it is preferred that the dimensions of the slave fermenters are similar to the dimensions of the master fermenter i.e. less than two time the dimensions of the master fermenter.

The Master Slave set-up provides for several benefits compared with stand alone fermenters: first, there is only need for fermenter controls on one fermenter which may reduce the need for instrumentation and/or operator time, and second it provide flexibility to the fermentation plant. For example, for a fermentation plant comprising a number of fermenters according to the invention, two or more fermenters can be combined in a master-slave setup in order to perform a fermentation in the desired volume for the particular fermentation. In this way is the production easily scalable and there is no need for fermenters in different sizes, different fermentation protocols depending of the size of the fermenter and separate instrumentation for each fermenter.

In a particular preferred embodiment the fermenter is made as a movable unit comprising the fermenter tank, one or more two-phase injectors, means for sterilization and optionally for cooling and fittings for connection to a circulation loop, substrate and other supplies, gas exhaust and optional probes, and the utilities for the fermenter is placed in a movable unit, e.g. a container, typically a container having the dimensions of a standard container used e.g. in shipping and transport industry.

In this connection the utilities for the fermenter is the necessary equipment for operating the fermenter and includes but is not limited to pump(s) for the circulation loop, means for generating and delivering sterile air for the ejector nozzles, inlets for nutrients, inoculation/seed materials, pH regulating chemicals, defoaming agents and other chemicals delivered during the fermentation; and outlets for sampling and/or harvesting the fermentation broth. Probes for measuring the conditions in the fermenter, such as temperature, oxygen saturation, pH, conductivity etc., may be provided in the circulation loop or directly in the fermenter.

The utilities may also comprise means to analyse the readings of the probes and means for controlling the supplies to the fermenter, typically a computer running control software. The invention also discloses the use of a fermenter according to the invention for growing one or more microorganisms for the production of one or more desired fermentation product(s).

The fermentation may be batch type fermentation, where all substrate and ingredients are provided from start; a fed-batch type fermentation, where the fermentation begin with a first amount of substrate in the fermenter and at a later time point after the fermentation process has started addition nutrients (feed) are added until the final volume in reached; or a continuous fermentation where nutrients are continuously supplied to the fermenter and fermentation broth is continuously removed from the fermenter. Such fermentation processes are well known in the art and the present invention is not limited by the use of any of these processes.

The desired product may be any product produced by microorganisms accumulating in the fermentation broth or it may even be the microorganisms itself. The fermentation product may be primary metabolites, secondary metabolites, proteins, vitamins, hormones and carbohydrates. The fermentation product is preferably selected among proteins, such as enzymes.

In one embodiment the fermentation product comprises an enzyme selected from the group of enzyme classes consisting of oxidoreductases (EC 1), transferases (EC 2), hydrolases (EC 3), lyases (EC 4), isomerases (EC 5), and ligases (EC 6).

In another embodiment the enzyme is an enzyme with an activity selected from the group of enzyme activities consisting of aminopeptidase, amylase, amyloglucosidase, mannanase, carbohydrase, carboxypeptidase, catalase, cellulase, chitinase, cutinase, cyclodextrin glycosyltransferase, deoxyribonuclease, esterase, galactosidase, beta-galactosidase, glucoamylase, glucose oxidase, glucosidase, haloperoxidase, hemicellulase, invertase, isomerase, laccase, ligase, lipase, lyase, mannosidase, oxidase, pectinase, peroxidase, phytase, phenoloxidase, polyphenoloxidase, protease, ribonuclease, transferase, transglutaminase, or xylanase.

### Recovery of the Compound of Interest

A further aspect of the invention concerns the downstream processing of the fermentation broth. After the fermentation process is ended, the compound of interest may be recovered from the fermentation broth, using standard technology developed for the compound of interest.

The relevant downstream processing technology to be applied depends on the nature of the compound of interest.

A process for the recovery of a compound of interest from a fermentation broth will typically (but is not limited to) involve some or all of the following steps:
1) pre-treatment of broth (e.g. flocculation)
2) removal of cells and other solid material from broth (primary separation)
3) filtration
4) concentration
5) stabilization and standardization.

Apart from the unit operations listed above, a number of other recovery procedures and steps may be applied, e.g., pH-adjustments, variation in temperature, crystallization, treatment of the solution comprising the compound of interest with active carbon, use of chromatography, and use of various adsorbents.

### PREFERRED EMBODIMENTS

The invention is further described by the following preferred embodiments:

### Embodiment 1

A fermenter for fermenting microorganisms for the production of a desired product, comprising a tank, one or more two-phase injectors for supplying oxygen localized in the lower part of the fermenter, at least one loop withdrawing fluid from the fermenter and circulating the fluid to provide liquid for the two-phase injectors and one or more inlets and one outlets.

### Embodiment 2

The fermenter of embodiment 1, wherein at least one of the one or more two-phase injectors are arranged so the injected stream is injected at an angle to a horizontal plane of 10-80°.

### Embodiment 3

The fermenter of embodiment 2, comprising at least two two-phase injectors, where at least one injector provides an injected stream going down, and at least one injector provides an injected stream going up

### Embodiment 4

The fermenter of embodiment 1 to 3, comprising at least 0.04 two-phase injector per m³ fermenter volume (=1 injector per 25 m³), preferably at least 0.05 two-phase injector per m³ fermenter volume, preferably at least 0.075 two-phase injector per m³ fermenter volume, preferably at least 0.1 two-phase injector per m³ fermenter volume, preferably at least 0.15 two-phase injector per m³ fermenter volume, preferably 0.20 two-phase injector per m³ fermenter volume and most preferred at least 0.25 two-phase injector per m³ fermenter volume.

### Embodiment 5

The fermenter according to any of the preceding embodiments, wherein the fermenter is capable of delivering at least 400 mg O₂ per hour per kg fermentation broth (mgO₂/kg/hr), preferably at least 500 mgO₂/kg/hr, preferably at least 600 mgO₂/kg/hr, preferably at least 700 mgO₂/kg/hr, e.g. at least 800 mgO₂/kg/hr, e.g. at least 900 mgO₂/kg/hr, e.g. at least 1000 mgO₂/kg/hr, e.g. at least 1200 mgO₂/kg/hr, e.g. at least 1500 mgO₂/kg/hr, e.g. at least 2000 mgO₂/kg/hr, e.g. at least 3000 mgO₂/kg/hr, e.g. at least 4000 mgO₂/kg/hr, e.g. at least 5000 mgO₂/kg/hr, e.g. at least 6000 mgO₂/kg/hr, e.g. at least 7000 mgO₂/kg/hr, e.g. at least 8000 mgO₂/kg/hr and most preferred at least 10000 mgO₂/kg/hr.

### Embodiment 6

The fermenter according to any of the preceding embodiments, wherein the circulation loop comprises one or more inlets and one or more outlets and is connected to a pump.

### Embodiment 7

The fermenter according to any of the preceding embodiments, wherein the fermenter is connected to a seed fermenter.

### Embodiment 8

The fermenter according to embodiment 7, wherein the seed fermenter has a volume in the range of 5-25% of the fermenter volume.

### Embodiment 9

The fermenter according to any of the preceding embodiments, further comprising means for cooling.

### Embodiment 10

The fermenter according to any of the preceding embodiments, further comprising one or more probes for measuring the conditions in the fermenter, such as temperature, pH, oxygen saturation, conductivity etc.

### Embodiment 11

The fermenter of embodiment 10, wherein the one or more probes for measuring the conditions in the fermenter is provided in the circulation loop.

### Embodiment 12

The fermenter according to any of the preceeding embodiments, wherein the volume is at least 50 liters, preferably at least 100 liters, preferably at least 500 liters, preferably at least 1000 liters, preferably at least 5000 liters, preferably at least 10000 liters, preferably at least 25000 liters, preferably at least 50000 liters, preferably at least 100000 liters or at least 250000 liters.

### Embodiment 13

The fermenter according to any of the preceeding embodiments, wherein the fermenter is sterilisable or CIP cleanable.

### Embodiment 14

A fermentation plant comprising one of more fermenters according to any of the embodiments 1-12, means for driving the flow in the circulation loops, supply of substrate, nutrients and air, means for analysing the readings of the probes connected to the fermenter, means for regulating pH and temperature and means for delivering additional compounds such as defoaming agents and other compounds required during the fermentation.

### Embodiment 15

The fermentation plant according to embodiment 14, wherein the means for driving the flow in the circulation loops, supply of substrate, nutrients and air, means for analysing the readings of the probes connected to the fermenter, means for regulating pH and temperature and means for delivering additional compounds such as defoaming agents and other compounds required during the fermentation is separated from the fermenter.

### Embodiment 16

The fermentation plant according to embodiment 14, wherein the means for driving the flow in the circulation loops, supply of substrate, nutrients and air, means for analysing the readings of the probes connected to the fermenter, means for regulating pH and temperature and means for delivering additional compounds such as defoaming agents and other compounds required during the fermentation is provided in a container placed adjacent to the one or more fermenters.

### Embodiment 17

A use of a fermenter according to any of the embodiments 1-13 or of a fermentation plant according to any of the embodiments 14-16 for growing one or more cells producing one or more fermentation products.

### Embodiment 18

The use of embodiment 17, wherein a first fermenter (Master fermenter) according to any of the embodiments 1-13 is connected to a second and optional subsequent fermenter(s) (Slave fermenter(s)) according to any of the embodiments 1-13 via the circulation loops.

### Embodiment 19

The use according to embodiment 17 or 18, wherein the use comprises, providing a substrate for growing the one or more cells in the fermenter, inoculating the fermenter with the one or more cells and growing the one or more cells until a desired amount of the one or more fermentation products is achieved.

### Embodiment 20

The use according to embodiment 19, further comprising feeding additional nutrients/substrate starting from a predetermined point.

### Embodiment 21

The use according to any of the embodiments 17-20, wherein the one or more cells are selected among prokaryotes selected among: Bacillus, Clostridium, Enterococcus, Geobacillus, Lactobacillus, Lactococcus, Oceanobacillus, Staphylococcus, Streptococcus, Streptomyces, Campylobacter, E. coli, Flavobacterium, Fusobacterium, Helicobacter, Ilyobacter, Neisseria, Pseudomonas, Salmonella, and Ureaplasma.

### Embodiment 22

The use according to embodiment 21, wherein the one or more cells are selected among Bacillus cell including, but not limited to, Bacillus alkalophilus, Bacillus altitudinis, Bacillus amyloliquefaciens, B. amyloliquefaciens subsp. plantarum, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus methylotrophicus, Bacillus pumilus, Bacillus safensis, Bacillus stearothermophilus, Bacillus subtilis, and Bacillus thuringiensis cells Streptococcus cell including, but not limited to, Streptococcus equisimilis, Streptococcus pyogenes, Streptococcus uberis, and Streptococcus equi subsp. Zooepidemicus cells and Streptomyces cell including, but not limited to, Streptomyces achromogenes, Streptomyces avermitilis, Streptomyces coelicolor, Streptomyces griseus, and Streptomyces lividans cells.

### Embodiment 23

The use according to any of the embodiments 17-20, wherein the one or more cells are selected among eukaryotes selected among: Candida, Hansenula, Kluyveromyces, Pichia, Saccharomyces, Schizosaccharomyces, Yarrowia, Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma cell

### Embodiment 24

The use according to embodiment 23, wherein the one or more cells are selected among: Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, Yarrowia lipolytica, Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminurn, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma Trichoderma longibrachiatum, Trichoderma reesei, and Trichoderma viride cell.

### Embodiment 25

The use according to any of the embodiments 17-24, wherein the one or more fermentation products are selected among primary metabolites, secondary metabolites, proteins, vitamins, hormones and carbohydrates.

### Embodiment 26

The use according to embodiment 25, wherein the one or more fermentation products are selected among proteins, such as enzymes.

### Embodiment 27

The use according to embodiment 26, wherein the enzymes are selected from the group of enzyme classes consisting of oxidoreductases (EC 1), transferases (EC 2), hydrolases (EC 3), lyases (EC 4), isomerases (EC 5), and ligases (EC 6).

### Embodiment 28

The use according to embodiment 27, wherein the enzymes are selected from the group of enzyme activities consisting of aminopeptidase, amylase, amyloglucosidase, mannanase, carbohydrase, carboxypeptidase, catalase, cellulase, chitinase, cutinase, cyclodextrin glycosyltransferase, deoxyribonuclease, esterase, galactosidase, beta-galactosidase, glucoamylase, glucose oxidase, glucosidase, haloperoxidase, hemicellulase, invertase, isomerase, laccase, ligase, lipase, lyase, mannosidase, oxidase, pectinase, peroxidase, phytase, phenoloxidase, polyphenoloxidase, protease, ribonuclease, transferase, transglutaminase, or xylanase.

### Embodiment 29

A fermentation installation comprising a first fermenter (Master fermenter) according to any of the embodiments 1-13, connected to a second and optional subsequent fermenter(s) (Slave fermenter(s)) according to any of the embodiments 1-13 via the circulation loops, so the two or more fermenters are controlled via the controller provided in connection with the first fermenter.

The invention is further illustrated in the following example which is not intended to be in any way limiting to the scope of the invention as claimed.

### Determination of Enzyme Activities

### Xylanase Assay:

The xylanase activity was determined using 0.2% AZCL-arabinoxylan as substrate in 0.01% Triton X-100 and 200 mM sodium phosphate pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6 buffer.

### Glucoamylase Activity (AGU):

The Glucoamylase Unit (AGU) is defined as the amount of enzyme, which hydrolyzes 1 micromole maltose per minute in a 0.1 M acetate buffer at an incubation temperature 37° C., a pH of 4.3, a maltose starting concentration of 100 mM, and a reaction time of 6 minutes, thereby generating alpha-D-glucose. The definition applies to an enzyme working range of 0.5-4.0 AGU/mL.

After incubation, the reaction may be stopped with NaOH and the amounts of glucose measured using the following two-step color reaction method: Glucose is phosphorylated by ATP, in a reaction catalyzed by hexokinase. The glucose-6-phosphate formed is oxidized to 6-phosphogluconate by glucose-6-phosphate dehydrogenase. In this same reaction, an equimolar amount of NAD+ is reduced to NADH with a resulting increase in absorbance at 340 nm. Reaction conditions are as specified in table 2 below:

TABLE 2
Color reaction

| Tris | approx. 35 mM | |
|---|---|---|
| ATP | 0.7 | mM |
| NAD⁺ | 0.7 | mM |
| Mg²⁺ | 1.8 | mM |
| Hexokinase | >850 | U/L |
| Glucose-6-P-DH | >850 | U/L |
| pH | approx. 7.8 | |
| Temperature | 37.0° C. ± 1.0° C. | |
| Reaction time | 420 | sec |
| Wavelength | 340 | nm |

### Example 1—Construction of Nozzle Fermenter

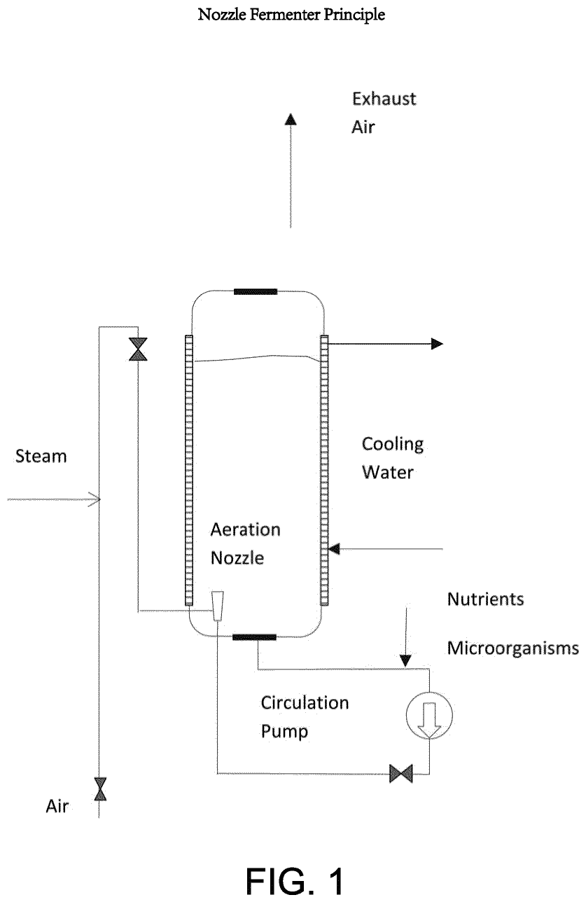

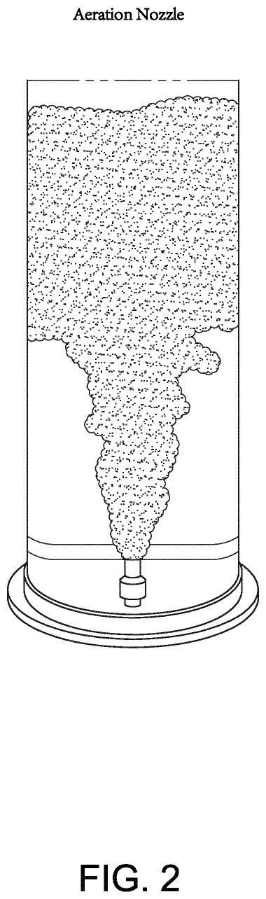

A fermenter according to the invention as disclosed in FIG. 1 was constructed using following parameters

TABLE 3

| Parameter | Fermenter |
|---|---|
| Fermenter Volume (m³) | 36 |
| Nozzles per volume | 0.22 |
| Height (m) | 11.9 |
| Diameter (m) | 2.0 |
| Air Pressure (barg) | 1.8 |
| Aeration - VVM | 0.1-1.0 |
| (volume/fermenter volume/min) | |
| Liquid Circulation | 0.5-2.8 |
| (volume/fermenter volume/h) | |

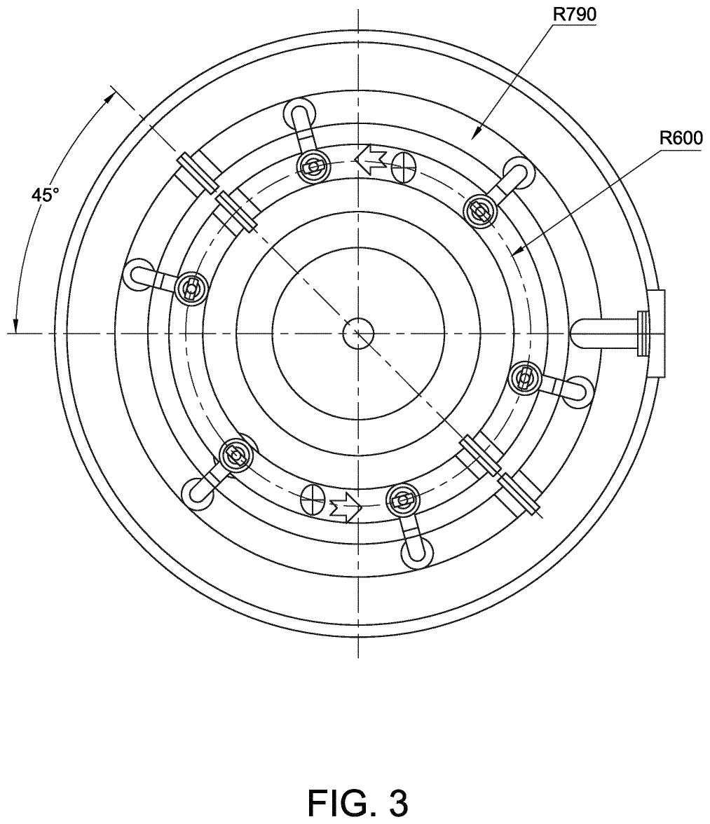

The nozzles (two phase injectors) were made in steel and had the dimensions of disclosed in Zlokarnik section 23.4.2. The Nozzle Arrangement for the fermenter is disclosed in FIG. 3.

### Example 2—Fungal Enzyme Fermentation by Use of Nozzle Fermenter and a Comparison with a Traditional Stirred Tank Reactor

The Nozzle Fermenter described in example 1 is used for production of cellulose degrading enzymes using a Trichoderma reesei strain.

This fermentation process used is limited by oxygen transfer and similar fermentation conditions are used for the Nozzle Fermenter and traditional Stirred Tank Reactors.

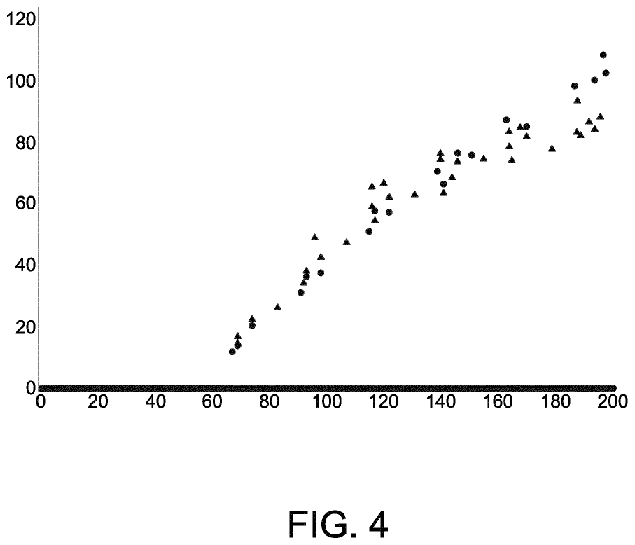

Relative Enzyme Titer Development as a function of Fermentation Time (h) for multiple batches is disclosed in FIG. 4 in comparison with STR fermenter.

As can be observed on the curve the nozzle fermenter has a similar performance until the last part of the fermentation, where it is levelling off compared to the Stirred Tank Reactor.

The costs for oxygen transfer is significantly lower as the pumping power is only approximately 10% of the agitation power used for STR fermenters.

### Example 3—Fermenting Trichoderma reesei for the Production of Xylanase

Xylanase fermentations using a Trichoderma reesei strain was used for this example using a standard industrial xylanase medium. The strains was fermented in the fermenter of the invention described in Example 1 (9 batches), in a 30 m³ stirred high power STR (6 batches) and in a 80 m³ medium-power STR (4 batches). The High power STR was run with a higher agitation rate providing a higher oxygen transfer rate but also added more heat to the fermenter that had to be removed using more cooling. The High power STR had an energy consumption for agitation that was 65% higher than for the medium power STR. Xylanase activity was measured at regular intervals in order to follow progress of the fermentations.

The results are summarized in FIG. 6, where the upper graph shown the performance of the high power STR reaching the highest titer. The middle graph shown the performance of the medium power STR reaching a titer of 88% of the titer for the High Power STR. The lowest graph shows the performance of the fermenter of the invention reaching a titer of 73% of the titer for the high Power STR.

The operating costs for the fermentation scaled to 80 m³ volume, including costs for raw materials, airflow, mixing, cooling etc; and the cost per produced xylanase unit were calculated: results are shown in table 4, where the figures for high power STR is set to 100%.

TABLE 4

| | High | Medium | Fermenter of the |
|---|---|---|---|
| | Power STR | Power STR | invention |
| Total operating costs | 100% | 88% | 71% |
| Operating cost per | 100% | 99% | 96% |
| xylanase unit | | | |

Thus the example shows that the fermenter of the invention can produce xylanase at a lower operating costs compared with the STR fermenter.

### Example 4.—Fermenting Aspergillus niger for the Production of Glucoamylase

Glucoamylase fermentations using an Aspergillus niger strain was used for this example using a standard industrial glucoamylase medium. The strain was fermented in the fermenter of the invention described in Example 1 and in a 80 m³ STR. The fermentation were performed for 150 h whereafter glucoamylase activity was determined. The fermentation of the fermenter of the invention was continued for additional 40 h to see if the fermenter could deliver the same yield as the STR if the fermentation time were extended.

The operating costs for the fermentation scaled to 80 m³ volume, including costs for raw materials, airflow, mixing, cooling etc; the yield and the cost per produced glucoamylase unit were calculated: results are shown in table 5, where the figures for the STR is set to 100%.

TABLE 5

| | | Fermenter of the | Fermenter of the |
|---|---|---|---|
| | STR | invention , 150 h | invention, 190 h |
| Yield | 100% | 78% | 100% |
| Total operating costs | 100% | 79% | 96% |
| Operating cost per | 100% | 101% | 96% |
| glucoamylase unit | | | |

This example shows that the fermenter of the invention can produce glucoamylase at the same operating costs as the STR and at a lower costs if the fermentation time is extended.

### Example 5—Fermenting Bacillus licheniformis for the Production of Subtilisin 309

A recombinant Bacillus licheniformis strain transformed with the gene encoding the subtilisin 309 (described in EP 396 608), using methods essentially as disclosed in WO 02/00907.

The strain was fermented in an industrial substrate for producing sublitisins. The strains was fermented in the fermenter of the invention described in Example 1, in a 30 m³ stirred high power STR and in a 160 m³ low-power STR. Protease activity was measured at regular intervals in order to follow progress of the fermentations.

The fermentation time for the fermenter of the invention was extended for additional 50% compared with STR.

The operating costs for the fermentation scaled to 80 m³ volume, including costs for raw materials, airflow, mixing, cooling etc; the yield and the cost per produced subtilisin unit were calculated: results are shown in table 6, where the figures for the high power STR is set to 100%.

TABLE 6

| | High | Low | Fermenter of the |
|---|---|---|---|
| | power STR | Power STR | invention |
| Titer | 100% | 91% | 92% |
| Total operating costs | 100% | 90% | 89% |
| Operating costs per | 100% | 97% | 96% |
| subtilisin unit | | | |

This example shows that the fermenter of the invention can produce subtilisin at lower operating costs as the STR and at a lower costs if the fermentation time is extended.

1-16. (canceled)

17. A fermenter for fermentation of microorganisms for the production of a desired product, comprising a tank, one or more two-phase injectors for supplying oxygen localized in the lower part of the fermenter, at least one loop withdrawing fluid from the fermenter and circulating the fluid to provide liquid for the two-phase injectors, one or more inlets and one or more outlets.

18. The fermenter of claim 17, wherein at least one of the one or more two-phase injectors are arranged so the injected stream is injected at an angle to a horizontal plane of 10-80°.

19. The fermenter of claim 18, comprising at least two two-phase injectors, where at least one injector provides an injected stream going down, and at least one injector provides an injected stream going up.

20. The fermenter of claim 17, comprising at least 0.04 two-phase injector per m³ fermenter volume (=1 injector per 25 m³).

21. The fermenter of claim 17, wherein the fermenter is capable of delivering at least 400 mg O₂ per hour per kg fermentation broth (mgO₂/kg/hr).

22. The fermenter of claim 17, wherein the circulation loop comprises one or more inlets and one or more outlets and is connected to a pump.

23. The fermenter of claim 17, further comprising one or more probes for measuring the conditions in the fermenter.

24. The fermenter of claim 17, comprising a volume of at least 50 liters.

25. The fermenter of claim 17, wherein the fermenter is sterilisable or CIP cleanable.

26. A method for producing one or more fermentation product, the method comprising growing one or more cells in the fermenter of claim 17.

27. The method of claim 26, wherein the one or more cells are selected from the prokaryote cells of Bacillus, Clostridium, Enterococcus, Geobacillus, Lactobacillus, Lactococcus, Oceanobacillus, Staphylococcus, Streptococcus, Streptomyces, Campylobacter, E. coli, Flavobacterium, Fusobacterium, Helicobacter, Ilyobacter, Neisseria, Pseudomonas, Salmonella, and Ureaplasma.

28. The method of claim 26, wherein the one or more cells are selected from eukaryote cells of Candida, Hansenula, Kluyveromyces, Pichia, Saccharomyces, Schizosaccharomyces, Yarrowia, Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma.

29. The method of claim 26, wherein the one or more fermentation products are selected from primary metabolites, secondary metabolites, proteins, vitamins, hormones and carbohydrates.

30. The method of claim 29, wherein the one or more fermentation products comprise one or more enzymes.

31. The method of claim 30, wherein the one or more enzymes comprise one or more aminopeptidases, amylases, amyloglucosidases, mannanases, carbohydrases, carboxypeptidases, catalases, cellulases, chitinases, cutinases, cyclodextrin glycosyltransferases, deoxyribonucleases, esterases, galactosidases, beta-galactosidases, glucoamylases, glucose oxidases, glucosidases, haloperoxidases, hemicellulases, invertases, isomerases, laccases, ligases, lipases, lyases, mannosidases, oxidases, pectinases, peroxidases, phytases, phenoloxidases, polyphenoloxidases, proteases, ribonucleases, transferases, transglutaminases, or xylanases.

32. A fermentation installation comprising a first fermenter according to claim 17, connected to a second fermenter according to claim 17, via one or more circulation loops, so the first and second fermenter are controlled in parallel by the first fermenter.
